(19) United States
(12) Patent Application Publication
(10) Pub. No.: US 2007/0276396 A1
(43) Pub. Date: Nov. 29, 2007

(54) Modular acetabular reamer

(75) Inventor: Thomas Francis McCarthy, Neshanic Station, NJ (US)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Howmedica Osteonics Corp., Mahwah, NJ (US)

(21) Appl. No.: 11/431,473

(22) Filed: May 10, 2006

(51) Int. Cl. A61B 17/00

(52) U.S. Cl. 606/81

(57) ABSTRACT

A modular part-spherical acetabular reamer including a first part-spherical element having an outer surface including a pole and a portion extending from the pole toward an equator of the reamer to a point intermediate the pole and the equator. The reamer also includes a second part-spherical element having an outer surface extending from the intermediate point towards the equator. Further, the reamer includes means for releasably coupling the first and second portions to form the modular part-spherical reamer.

### BACKGROUND OF THE INVENTION

The present invention relates to surgical instruments and, in particular to acetabular reamers.

An acetabular reamer having a reamer head is used in hip replacement surgery for re-forming the hip socket, or acetabulum, in preparation for implanting a prosthetic component, such as an acetabular cup or socket. To insure a proper fit of the prosthetic device, the deteriorated or diseased bone and cartilage needs to be cut or shaved away to healthy bone tissue so that the reamed acetabulum matches the contours of the prosthetic to be fitted.

In a typical hip replacement procedure, including an acetabular implant, a surgeon makes an incision in the hip area, displaces the existing hip joint, shapes the acetabulum with the reamer to receive a metallic or plastic prosthetic socket, inserts the prosthetic socket, replaces the ball of the femur with a prosthetic ball, and inserts the prosthetic ball into the prosthetic socket to complete the operation.

Typically, a reamer head is comprised of a continuous exterior surface that requires a large incision in the skin of the patient in order to place the reamer head adjacent the socket to be reamed. Such a reamer is shown in U.S. Pat. Nos. 5,658,290, 6,106,536 and 6,702,819. Unfortunately, the size of the incision, effects the time that is required for recovery of the operation with a larger size incision increasing the recovery time. In addition, a larger incision also makes the patient more susceptible to infection and increases the time frame that disease may be introduced into the body via the incision.

### SUMMARY OF THE INVENTION

The present invention is directed to surgical instruments and implants. In one aspect of the present invention, a rotary surgical reamer assembly for moving bone and tissue from a bone joint include a dome and a body. The dome may include an exterior and an interior as well as a plurality of cutting sites disposed on the exterior and a plurality of apertures adjacent to the cutting sites. The body preferably includes a base, a ridge and at least one column connecting the base to the ridge. In one aspect of the present invention, the dome is attached to the ridge.

The assembly also preferably includes a circumferential ridge having an exterior and interior, a first edge, a second edge and an opening extending from the first edge to the second edge. The circumferential ring includes a plurality of cutting sites disposed on the exterior and a plurality of apertures adjacent to the cutting sites. The first edge of the circumferential ring is adjacent to the dome and the second edge of the circumferential ring is adjacent to the base of the body. Preferably, the exterior of the dome and the exterior of the circumferential ring form a substantially continuous wall.

In one aspect of the present invention, the dome and the circumferential ring are part-spherical and in combination form a substantially hemispherical shape. The dome may include a pole further comprising a polar axis passing through the pole. The exterior of the dome and the exterior of the circumferential ring are preferably substantially curvilinear relative to the polar axis.

In one aspect, the interior of the circumferential ring may include a first portion rectilinear relative to the polar axis such that when the circumferential ring is disposed on the body, the first portion engages the at least one column to thereby prevent rotation of the circumferential ring relative to the body.

The dome may be remote from the base of the body at a sufficient distance to permit the dome and the body to be placed through an incision concurrently without causing the cutting sites of the dome to engage soft tissue surrounding the incision, as compared to when the circumferential ring and the body are passed through the incision concurrently where the cutting sites would contact the soft tissue surrounding the incision.

In one aspect of the present invention, a method of removing bone and soft tissue from a bone joint may include providing a first portion of a surgical reamer. The first portion having a first cutting surface for removing bone and soft tissue. The method also comprising providing a second portion of a surgical reamer that also includes cutting surfaces for removing bone and soft tissue. After making an incision at a portion of a body to allow access to a bone joint, the first portion is placed through the incision and subsequently the second portion is place through the incision and into the vicinity of the bone joint. Once the first portion and second portion are placed within the vicinity of the bone joint, they are assembled together such that the first cutting surface and the second cutting surface form a substantially single cutting surface.

In one aspect of the present invention, a method of removing bone and soft tissue from a bone joint includes making an incision into the skin of a patient to allow access to a desired location. The incision has dimensions that permit a first portion and a second portion of a reamer to pass through the incision and into the desired location, without a cutting surface of the first portion or second portion coming into contact with the skin surrounding the incision. The method also includes assembling the first portion of the reamer to the second portion of the reamer wherein the resulting assembly has overall dimensions larger than the dimensions of the incision such that if the resulting assembly was passed through the incision, the cutting surface of either the first portion or the second portion would come in contact with the skin surrounding the incision.

In another aspect of the present invention, an orthopedic implant is provided having a core with an interior surface, an exterior surface, a first end and an apex. The core also includes an engagement element. The implant further includes a ring having an interior surface, an exterior surface, a first edge, a second edge and an opening extending from the first edge to the second edge. The ring also having an engagement element, which corresponds to the engagement element of the core. The opening of the ring being sized to receive the core such that the apex of the core may be passed through the opening until the first end of the core is adjacent to the first end of the ring. When the first end of the core is adjacent to the first end of the ring, the engagement element of the core may be mated with the engagement element of the ring to thereby lock the core to the ring. In one embodiment the orthopedic implant described above is an acetabular shell implant.

### BRIEF DESCRIPTION OF THE DRAWINGS

FIG. 1 illustrates one embodiment of an acetabular reamer assembly according to the present invention;

FIG. 2 is a side view of a first component of the acetabular reamer assembly shown in FIG. 1;

FIG. 3 is a top view of the first component shown in FIG. 2;

FIG. 4 is a bottom view of the first component shown in FIG. 2;

FIG. 5 is a side view of second component of the acetabular reamer shown in FIG. 1;

FIG. 6 is a top view of the second component shown in FIG. 5;

FIG. 7 illustrates the second component of FIG. 5 being inserted into a patient;

FIG. 8 illustrates another aspect of the present invention relating for a two-part first component;

FIG. 9 illustrates an embodiment of an acetabulum shell according to the present invention.

### DETAILED DESCRIPTION

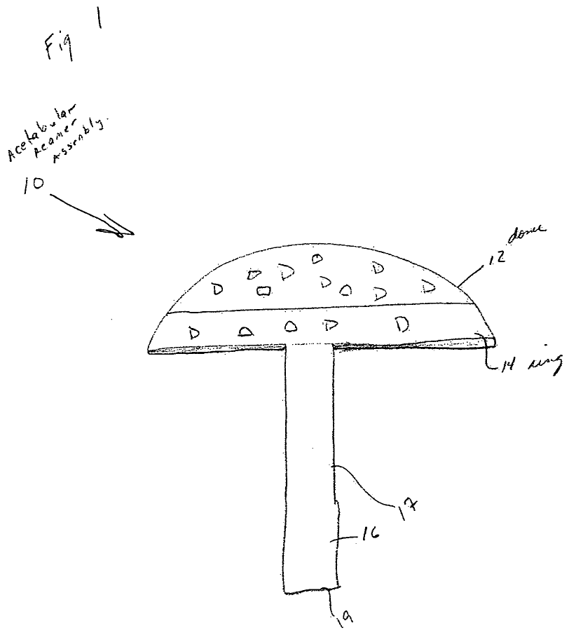

The present invention relates to a surgical reamer assembly in the form of an acetabular reamer assembly 10 as shown in FIG. 1. An acetabular reamer assembly 10 is generally useful for removing bone and tissue from a joint socket when performing reconstructive surgery, to facilitate the installation of a prosthetic device or to repair the damaged bone.

In one aspect of the present invention, the acetabular reamer assembly 10 preferably includes a first component having a body (not shown in FIG. 1), coupled to a dome 12 and a second component comprising a ring 14. The dome 12 and the ring 14 in combination define the cutting surface of the acetabular reamer assembly 10. A handle 16 is attached to the body and preferably extends transversely from the dome 12 as will be described below. The adjustment handle 16 may include a drive shaft 17 having a drive end 19 that is attached to a rotational driver such as a surgical drill (not shown). The surgical drill provides rotational movement to the drive shaft 17, which in turn causes the dome 12 and the ring 14 to rotate. Such surgical drills are known to those in the art as for example surgical drills included in U.S. Patent Publication Nos. 2004/0087958, 2004/0153080, and 2005/0216022, the disclosures of which are hereby incorporated by reference herein.

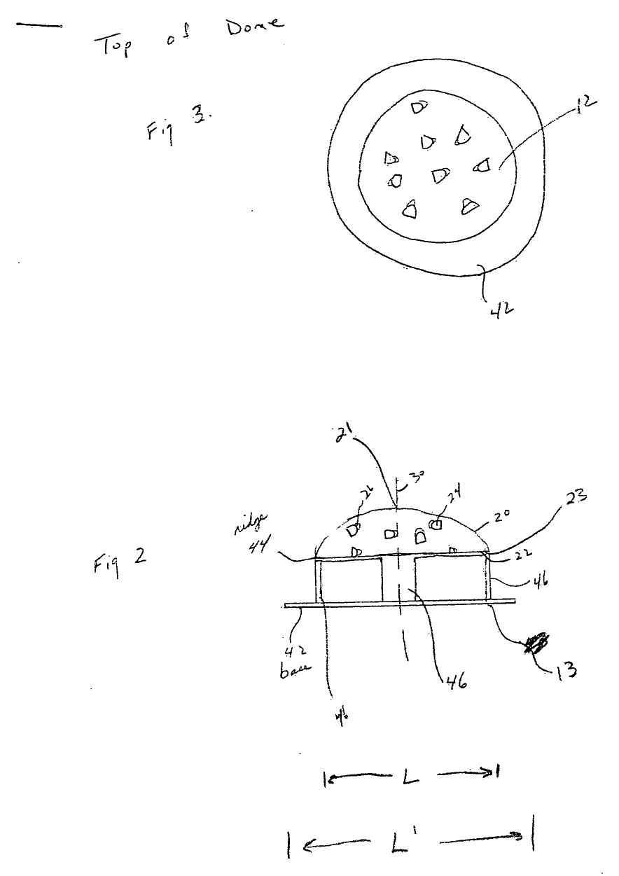

With reference to FIGS. 2-4, various aspects of the dome 12 will now be described. The dome 12 is preferably formed from stainless steel and includes an exterior surface 20 and an interior surface 22. A plurality of cutting sites 24 is disposed along the exterior surface 20 of the dome 12. The cutting sites 24 may comprise a plurality of cupped cutters distributed along the exterior surface 20 and oriented to face a rotational cutting direction, as known in the art. An aperture 26, extending from the exterior surface 20 to the interior surface 22 may be positioned next to each cutting site 24. The apertures 26 enable any material cut by the cutting sites 24 to pass from the exterior surface 20 of the dome 12 through the aperture and into a void below the dome. The cutting sites 24 preferably have sharp edges that extend beyond the exterior surface 20 of the dome 12. The cutting sites 24 may be thought of as a plurality of grating holes, having a raised sharpened edge on one side of the hole facing the cutting direction for cutting and removal of bone in the acetabular region.

The dome 12 preferably has an arcuate shape similar to a portion of a sphere that includes an apex of the sphere. Thus, as shown in FIG. 2, the dome 12 has an apex 21 and a bottom ridge 23 opposite the apex. A polar axis 30 passes through the apex of the dome 12 and a center of the dome. The arcuate dome 12 extends outwardly in a direction towards the equator of the sphere from the apex 21. The dome has a maximum diameter L extending from a first point along the bottom ridge 23 to a second point along the ridge, the first point being 180° away from the second point along the bottom ridge but on the opposite side (180° along the circumference). In the preferred embodiment the spherical dome extends about 30° latitude with respect to an equator at approximately 90° from the apex 21.

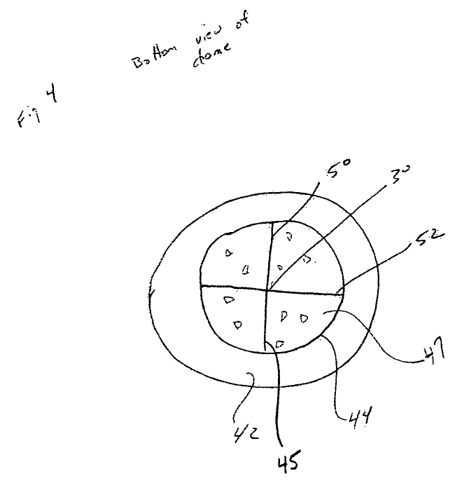

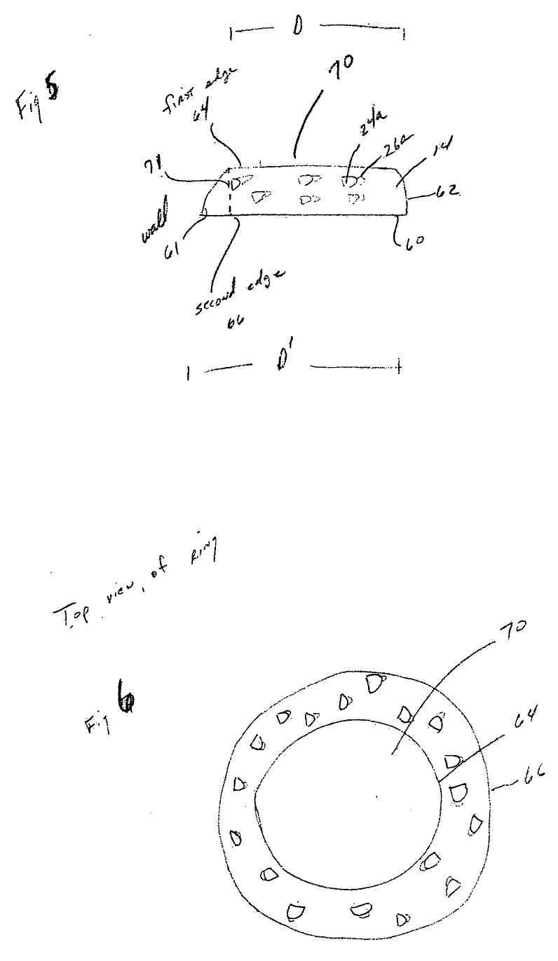

The dome 12 may be permanently or temporarily affixed to the main frame body 13. In one aspect of the present invention, the main frame body 13 includes a base 42, a ridge 44, remote from the base, and a plurality of connecting walls such as columns 46 extending from the base to the ridge. As shown in FIG. 3, the preferred base 42 is generally circular and similarly, as shown in FIGS. 3 and 4, the ridge 44 is also circular. Both the base 42 and the ridge 44, preferably extend about axis 30, which also passes through a center of body 13, such that base 42 and ridge 44 are concentric with one another. The ridge 44 preferably has a maximum diameter that is substantially equal to the maximum diameter L of the dome 12. However, the base 42 has a maximum diameter L′ that is greater than the maximum diameter L of the dome 12. The lengths all being measured from a first point along a circumference of the particular element to a second point along the circumference but 180° away from the first point.

Also as shown in FIG. 4, a cross-bar system 45 is provided within an internal void 47 of the body 13. The cross-bar system 45 includes a first bar 50 and a second bar 52, which enable the handle 16 to be attached to the body 13. As shown in the figure, the first bar 50 extends from a point along the edge of the ridge 44 through the center of the device, as defined by axis 30 to an opposite edge of the ridge. The second bar 52, similarly extends from one edge of the ridge 44 to an opposite edge of the ridge and also passes through the center. Thus, the first bar 50 and the second bar 52 are preferably perpendicular to one another.

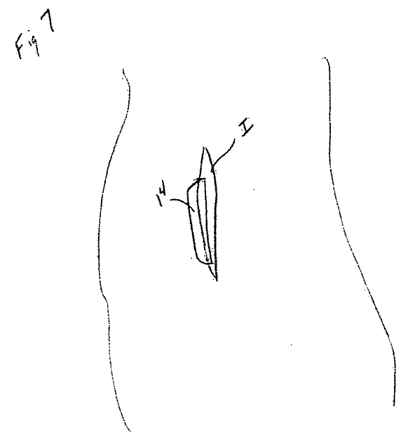

Another aspect of the present invention is ring 14, shown in FIGS. 5 and 6. Similar to dome 12, ring 14 includes an interior surface 60, exterior surface 62 and is formed from stainless steel. Also similar to dome surface 12, ring 14 includes a plurality of cutting sites 24A disposed along its exterior surface 62 and a plurality of apertures 26A positioned adjacent the cutting sites. The apertures 26A extend from the exterior surface 62 to the interior surface 60 of the ring and allow material cut by the cutting sites 26A to pass from the exterior surface 62 of the ring to a void within the center of the ring. The ring 14 is preferably arcuate and concaved relative to axis 30 at least along its exterior surface 62. The ring 14 also includes a first circular edge 64 having a first diameter D and a second circular edge 66 having a second diameter D′ remote from the first circular edge. The edges 64 and 66 define the boundaries of ring 14. In a preferred embodiment, the maximum diameter L of the ridge 44 of the body has a dimension that is slightly less the dimension of the first diameter D of the first circular edge 64. And the maximum diameter of the base 42 has a dimension that is slightly larger than the dimension of the second diameter D′ of the second circular edge 66. The first circular edge 64 and the second circular edge 66 are preferably parallel to one another with the outer surface of the ring being part-spherical.

The ring 14 is situated about axis 30 and includes an opening 70 extending from the first edge 64 to the second circular edge 66. In one preferred embodiment, the opening 70 has a larger diameter adjacent to second circular edge 66 than its diameter adjacent to first circular edge 64.

As shown in FIG. 5 in shadow, the interior surface 60 of the ring 14 preferably includes at least one wall 71 that is perpendicular to first circular edge 64 or at least rectilinear to the edge. When the dome 12 and particularly the body 13 are assembled to the ring 14, the wall 71 interacts with at least one column 46 to thereby prevent the ring 14 from rotating about the body, as will be described below.

In a method of assembling the acetabular reamer assembly 10, the dome 12 is attached to the body 13, either integrally or modularly such as being held on by screws or a taper lock. The dome 12 may also be snap-fitted to the body 13 or each element may have a structure that corresponds to the other structure, which permits engagement between the two elements but does not permit rotation between the two. The ring 14 is placed over the dome 12 with the apex 21 of the dome being received within the opening 70 of the ring adjacent to second circular edge 66. The dome 12 and body 13 are continually translated through the opening 70 until the entire dome 12 extends outwardly past opening 70 adjacent first circular edge 64. With the dome 12 positioned atop of the first circular edge 64 of the ring 14, most of the body 13 is retained within opening 70 of ring 14. But the ridge 44 of the body 13 is adjacent the first circular edge 64 of the ring 14 and the second circular edge 66 of the ring 14 is adjacent base 42 of the body. Since the base 42 has a larger maximum diameter L′ than the length of the second diameter D′, the body 13 can not pass entirely though the opening 70. When assembled together, the dome 12 and ring 14 form a hemispherical body having a plurality of cutting sites 24 and 24A and a plurality of apertures 26 and 26A extending about their respective exterior surfaces, as shown in FIG. 1. The second circular edge 66 is approximately equivalent to an equator of a sphere.

In this configuration, the acetabular reamer assembly 10 is ready to be employed to ream out an acetabulum such that an acetabular shell may be positioned correctly during reconstructive surgery.

In order to prevent the ring 14 from being able to rotate about the body 13 when the acetabular reamer assembly is employed and more specifically when a force causes the ring 14 and the dome 12 to rotate about axis 30, an engaging mechanism may be provided as alluded to before. For instance, when assembling the ring 14 and the dome 12 together, the wall 71 of the ring 14 may be aligned with one of the columns 46 such that these two linear elements confront each other. Since the wall 71 and the column 46 are parallel or at least rectilinear to axis 30, and are proximate one another when the acetabular reamer assembly is assembled, once coupled together, they do not permit the ring 14 to rotate about the body 13 of the acetabular reamer assembly 10. Therefore, when the acetabular reamer assembly 10 is employed and a rotational force is applied to the body 13 via a surgical rotary hand piece, the ring 14, and dome 12 also rotate simultaneously. The engagement mechanism prevents unwanted motion of the ring 14 relative to the body 13.

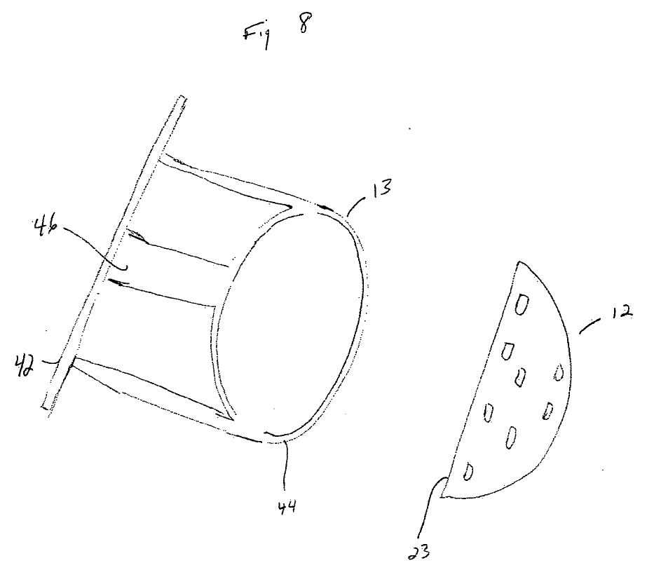

In a method of use, the ring 14 and dome 12 are placed through an incision in the skin of a patient separately. After a surgeon determines an incision location on the patient, he next slices the skin to create the incision I, as shown in FIG. 7. The ring 14 is then placed sideways through the incision. A sideways orientation of the ring 14 refers to an orientation wherein the plane of the first edge 64 and the plane of the second edge 66 are more parallel to the longitudinal direction of the incision I, than they are perpendicular, as shown in FIG. 7. Once the ring 14 has been positioned in-situ, the ring is rotated such that the opening 70 and more specifically the second circular edge 66 of the ring faces the incision opening. Since the ring 14, when orientated sideways, has a relatively narrow width, the size of the incision may be smaller than usually required to insert an integral cutting surface of a conventional acetabular reamer.

Next, the dome 12 and body 13, which are already assembled together, are translated through the incision I. As with the ring 14, during introduction through the incision I the plane of the base of the dome/body are oriented sideways with a plane that is tangential to the apex 21 of the dome and the base 42 of the body being more parallel to the longitudinal direction of the incision I, than they are perpendicular. When translating the dome/body 12,13 through the incision the base 42 may be used to pry the walls of the incision I apart in order to enlarge the incision opening. Since the skin is generally flexible, this can be accomplished without any further tearing of the skin. Once again, because the dome/body 12, 13 only has cutting sites 24 along one portion of the combination component, i.e., dome 12 and body 13, and more specifically does not have cutting sites proximate the base 42 of the body, the size of the incision required to place the dome/body component in-situ is smaller than the size of an incision required to place a reamer having a continuous hemispherical cutting surface. This prevents the cutters from damaging the soft tissue.

Thus, since the acetabular reamer assembly 10 is positioned within the body in two stages, and specifically the cutting sites 24, 24A are separately inserted, the incision I may by smaller than required for conventional hemispherical reamers. This is because conventional hemispherical reamers include a continuous exterior surface with cutting sites. As the conventional reamer is inserted through an incision, the incision must be sufficiently wide to prevent the skin from being cut by the cutting sites. This is particularly problematic where a cross section of the reamer is at its largest diameter such as at a position nearest the base of the reamer. This is because as the reamer is inserted sideways, the incision must be long enough to receive the maximum diameter of the reamer but also wide enough to receive the width of the reamer. Thereby requiring a relatively large incision.

The present invention avoids this by dissecting the cutting sites 24, 24A into two distinct halves, the dome 12 and the ring 14. When oriented sideways, the ring 14 may have a maximum diameter that is equal to the diameter of a conventional hemispherical reamer also oriented sideways but the width of the ring is less. The combination of the dome 12 and body 13, when oriented sideways, has a width equal to the width of a conventional reamer but a maximum diameter that is less. And since the combination of the dome 12 and body 13 does not include cutting sites positioned proximate the end of the body that is remote from the dome, to insert the dome/body 12,13, the base 42 of the body maybe placed against the skin positioned adjacent a longitudinal side of the incision I. The body may then be used to pry open the flexible skin such that the dome/body 12,13 may be inserted therethrough. Because all of the cutting sites 24 are proximate only the ends of only one side of the incision, the likelihood that the cutting sites may contact the skin is reduced. Plus the requirement of greatly stretching the skin or having a larger incision is eliminated, as compared to an incision required for conventional hemispherical reamers. This configuration enables the dome/body 12, 13 to be received through an incision while minimizing the risk that a cutting site will tear the skin around an incision.

Once the dome/body 12, 13 are placed in-situ, they are rotated such that the apex 21 of the dome is aligned with the opening 70 of the ring 14. Also, if an engagement member is provided such as discussed above, it may be correctly aligned. For instance, a column 46 of the body may be aligned with the wall 71 of the ring 14. The dome/body 12, 13 is translated through the opening 70 until the entire dome 12 extends outwardly from the opening with the bottom ridge 23 of the dome positioned adjacent the first circular edge 64 of the ring. The wall 71 and column 46 may not only prevent unwanted movement between the ring 14 and body 12 but may also provide a key-way system, that only permits the components to be coupled together in a specific spatial relationship. In other words, the ring 14 and body 13 can not be coupled together unless the wall 71 and a column 46 are aligned.

Once the elements are assembled together and the handle 16 is coupled to the cross-bar system (the handle is preferably coupled to the body during insertion of the dome and the body), a rotary power device may be connected to the handle so as to provide rotational movement to the handle 16 and subsequently to the dome 12 and ring 14.

As the dome 12 and ring 14 rotate about axis 30, the cutting sites 24, 24A are brought into proximity of the bone and soft tissue to be reamed to thereby begin the process of creating a cavity in the bone.

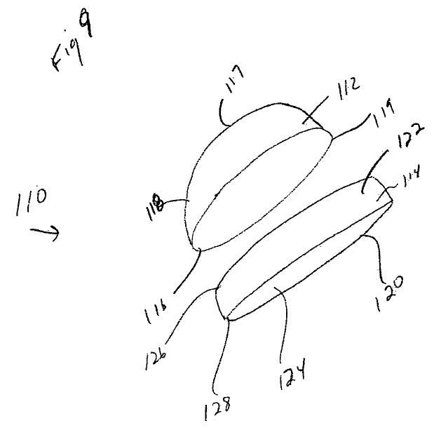

In another aspect of the present invention, as already alluded to, the dome 12 may be coupled to the body 13 in a removable manner. Thus, as shown in FIG. 8, the dome 12 is shown disassembled from the body 13. To fit the two elements together, the bottom ridge 23 of the dome may be removably attached or permanently attached to ridge 44 of the body 13 such as by snap fitting, welding, using various catch-mechanisms known to those in the art or the like.

Although the present invention has been described in conjunction with an acetubular reamer assembly, the concept of the invention may also be adapted for other purposes. For instance, an acetabulum implant shell 110 may be dissected in half to form a core 112 and a ring 114, as shown in FIG. 9. The core includes an interior surface 116, an exterior surface 118, an apex 117 and a circular edge 119. Similarly, the ring 114 includes an interior surface 120, an exterior surface 122 and an opening 124 extending from a first circular edge 126 of the ring to a second circular edge 128. As with the acetabular reamer assembly 10, the components of the acetabulum shell implant 110 may be inserted in a two-step process. This reduces the size of the incision required for inserting the shell, as compared to unitary shells.

In one method, after the acetabulum has been reamed, the ring 114 is inserted through an incision and placed adjacent the acetabulum. Next, the core 12 is inserted through the incision and subsequently through the opening 124 of the ring 114. The core 112 is translated entirely through the opening 124 until the circular edge 119 of the core 112 is adjacent the first circular edge 126 of the ring 114. In addition, the exterior surface 118 of the core 112 is positioned adjacent the acetabulum. Preferably, the interior surfaces, 116, 124 of the core 112 and ring 114 form a substantially smooth and continuous surface such that a prosthesis placed adjacent the interior surfaces can angulate and rotate smoothly against the surfaces.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A rotary surgical reamer assembly for removing bone and tissue for a bone joint, the assembly comprising:
a dome having an exterior and an interior, said dome wall including a plurality of cutting sites disposed on said exterior and a plurality of apertures adjacent said cutting sites, said apertures extending from said exterior to said interior;
a body including a base, a ridge and at least one column connecting said base to said ridge, said dome being attached to said ridge; and
a circumferential ring including an exterior, an interior, a first edge, a second edge and an opening extending from said first edge to said second edge, said circumferential ring including a plurality of cutting sites disposed on said exterior and a plurality of apertures adjacent said cutting sites, said apertures extending from said exterior to said interior;
wherein said first edge of said circumferential ring is adjacent said dome and said second edge of said circumferential ring is adjacent said base of said body, said exterior of said dome and said exterior of said circumferential ring forming a substantially continuous wall.

2. The assembly of claim 1, wherein said dome and said circumferential ring are part-spherical and in combination form a substantially hemispherical shape.

3. The assembly of claim 2, wherein said dome includes a pole, further comprising a polar axis passing through said pole, said exterior of said dome and said exterior of said circumferential ring being substantially curvilinear relative to said polar axis, said at least one column of said body being rectilinear relative to said polar axis.

4. The assembly of claim 3, wherein said interior of said circumferential ring includes a first portion rectilinear relative to said polar axis such that when said circumferential ring is disposed on said body said first portion engages said at least one column to thereby prevent rotation of said circumferential ring relative to said body.

5. The assembly of claim 1, wherein said dome is remote from said base of said body at a sufficient distance to permit said dome and said body to be placed through an incision concurrently without causing said cutting sites of said dome to engage soft tissue surrounding the incision, wherein the cutting sites of said circumferential ring do engage the soft tissue surrounding the incision when the circumferential and body are passed through the incision concurrently.

6. A method of removing bone and soft tissue from a bone joint, the method comprising:
providing a first portion of a surgical reamer, said first portion having a first cutting surface for removing bone and soft tissue;
providing a second portion of a surgical reamer, said second portion having a second cutting surface for removing bone and soft tissue;
making an incision at a portion of a body to allow access to a bone joint;
placing said first portion through said incision and into the vicinity of the bone joint;
placing said second portion through said incision and into the vicinity of the bone joint; and
assembling said first portion to said second portion while in the vicinity of the bone joint such that said first cutting surface and said second cutting surface form a substantially single cutting surface.

7. The method according to claim 6, wherein said second portion includes a dome wall having a plurality of cutting sites and a body attached to said dome wall.

8. The method according to claim 7, wherein said first portion includes a circumferential ring having a plurality of cutting sites and an opening extending from a first edge of said ring to a second edge of said ring, wherein said assembling step includes placing said ring over said dome wall such that said dome wall translates through said opening and an interior of said ring engages said body.

9. The method according to claim 8, wherein when said first portion is assembled to said second portion a cutting surface have a substantially hemispherical shape is formed.

10. A method of removing bone and soft tissue from a bone joint, the method comprising:
making an incision into the skin of a patient to allow access to a desired location, said incision having dimensions that permit a first portion and a second portion of a reamer to pass through said incision and into said desired location, without a cutting surface of said first portion or said second portion coming into contact with the skin surrounding the incision;
assembling said first portion of said reamer to said second portion of said reamer, wherein said resulting assembly has overall dimensions larger than the dimensions of said incision such that if said resulting assembly was passed through said incision, said cutting surfaces of either said first portion or said second portion would come in contact with the skin surrounding the incision.

11. A modular part-spherical acetabular reamer comprising:
a first part-spherical element having an outer surface including a pole and a portion extending from the pole toward an equator of the reamer to a point intermediate the pole and the equator;
a second part-spherical element having an outer surface extending from said intermediate point towards said equator; and
means for releasably coupling said first and second portions to form said modular part-spherical reamer.

12. An orthopedic implant comprising:
a core having an interior surface, an exterior surface, a first end and apex, said core also having an engagement element; and
a ring having an interior surface, an exterior surface, a first edge, a second edge and an opening extending from said first edge to said second edge, said ring also having an engagement element which corresponds to said engagement element of said core;
wherein said opening is sized to receive said core such that said apex of said core is passed through said opening until said first end of said core is adjacent said first edge of said ring;
further wherein when said first end of said core is adjacent said first edge of said ring said engagement element of said core may be mated with said engagement element of said ring to thereby lock said core to said ring.

13. The orthopedic implant according to claim 12, wherein said core and said ring are part of an acetabular shell implant.

14. The orthopedic implant according to claim 14, wherein said exterior surface of said core and said exterior surface of said ring form a substantially smooth surface when said core is engaged with said ring.

15. The orthopedic implant according to claim 14, wherein said core and said ring are placed through an incision of a patient separately and assembled in-situ.
